(19) United States
(12) Patent Application Publication
(10) Pub. No.: US 2022/0168445 A1
(43) Pub. Date: Jun. 2, 2022

(54) FOCUSED ULTRASOUND FOR NON-INVASIVE FOCAL GENE DELIVERY TO THE MAMMALIAN BRAIN

(71) Applicants: Cornell University, Ithaca, NY (US); The Rockefeller University, New York, NY (US)

(72) Inventors: Michael G. Kaplitt, New York, NY (US); Mihaela Stavarache, New York, NY (US)

(21) Appl. No.: 17/442,790

(22) PCT Filed: Mar. 27, 2020
(86) PCT No.: PCT/US2020/025486

§ 371 Date: Sep. 24, 2021

Related U.S. Application Data
(60) Provisional application No. 62/824,711, filed on Mar. 27, 2019.

(51) Int. Cl. A61K 48/00; A61N 7/00; G01R 33/56; C12N 15/87

(52) U.S. Cl. CPC A61K 48/005; A61N 7/00; G01R 33/5601; C12N 2750/14143; C12N 15/87; A61N 2007/0039; A61N 2007/0021; A61K 48/0008

(57) ABSTRACT

Methods of employing non-oncolytic viruses or plasmids and focused ultrasound are provided. Specifically, the disclosure provides methods for ultrasound-mediated non-invasive delivery of gene therapy agents to the central nervous system, wherein magnetic resonance (MR) guided focused ultrasound is used, optionally in combination with microbubbles, to facilitate gene delivery to a particular region of the brain.

### CROSS-REFERENCE TO RELATED APPLICATIONS

This application claims the benefit of the filing date of U.S. application No. 62/824,711, filed on Mar. 27, 2019, the disclosure of which is incorporated by reference herein.

### BACKGROUND

Gene therapy has proven to be safe and effective for a variety of neurological and oncological diseases in both preclinical studies and in human trials. One limitation, however, has been the continued need for direct injection of gene therapy agents into the brain. This creates risks of invasive surgery, such as hemorrhage. It also can be very difficult to tailor the delivery of agents to optimally cover desired brain targets with direct infusion, since the direction and coverage of fluid flow is limited by the need to deliver from a single point at the end of an infusion catheter. For tumors of the central nervous system, this is particularly problematic since tumor recurrence following surgery and adjunctive therapy is usually due to cells which have invaded the otherwise normal brain tissue surrounding the visible mass and direct infusion of gene therapy agents into the surrounding normal brain tissue to attack invading tumor cells can be difficult and highly inefficient due to altered tissue resistance and flow dynamics in the presence of a resection cavity. Systemic delivery of certain gene therapy agents can lead to transduction in the brain, due to the ability to cross the blood-brain barrier (BBB). However, these are not only generally inefficient, but they also cannot be targeted to particular areas of the brain. For many applications, for example in neurodegenerative and psychiatric diseases, global delivery of genes throughout the brain and spinal cord would not be optimal since they could cause untoward effects from genetically modifying regions of the brain or spinal cord which might have effects contrary to the therapeutic goals of gene delivery to the desired target. Systemic delivery of gene therapy agents may also result in transduction of peripheral organs, which can lead to adverse effects due to unnecessary and undesired gene expression in peripheral organs.

### SUMMARY

The present disclosure provides for gene therapy for neurological, psychiatric and neuro-oncological diseases which permits delivery of genes to targeted regions of the central nervous system without direct invasion of the brain. Specifically, the disclosure provides materials and methods useful for ultrasound-mediated non-invasive delivery of gene therapy agents to the central nervous system. In one example, magnetic resonance (MR) guided focused ultrasound is used, optionally in combination with microbubbles, to facilitate gene delivery to a particular region of the brain. In one embodiment, microbubbles are injected intravenously immediately before or during the procedure to delivery ultrasound to one or more central nervous system target(s). In one embodiment, microbubbles are delivered simultaneously with the gene therapy vector.

Surgical infusion of gene therapy vectors has provided opportunities for biological manipulation of specific brain circuits in both animal models and human patients. Transient focal opening of the blood brain barrier (BBB) by MR-guided focused ultrasound (MRgFUS) may allow for non-invasive CNS gene therapy to target precise brain regions. The data herein pertain to the efficiency, safety, and long-term stability of MRgFUS-mediated non-invasive gene therapy in the mammalian brain. In one embodiment focused ultrasound under the control of magnetic resonance imaging (MRI) in combination with microbubbles (MB) formed of lipid-coated gas microspheres was applied to rat striatum, followed by intravenous infusion of a adeno-associated virus serotype 1/2 vector (AAV1/2) expressing green fluorescent protein (GFP) as a marker. Following recovery, animals were followed from several hours up to 15 months. Immunostaining for GFP quantified transduction efficiency and stability of expression. Quantification of neuronal markers were used to determine histological safety over time, while inflammatory markers were examined for evidence of immune responses. Transitory disruption of the BBB by MRgFUS resulted in efficient delivery of the AAV1/2 vector to the targeted rodent striatum, with 50-75% of striatal neurons transduced on average. GFP transgene expression appeared to be stable over extended periods of time, from two weeks to 6 months, with evidence of ongoing stable expression out to 16 months in a smaller cohort of animals. Evidence of substantial toxicity, tissue injury or neuronal loss was not observed. While transient inflammation from BBB disruption alone was noted for the first few days, consistent with prior observations, no evidence of brain inflammation was observed from three weeks to 6 months following MRgFUS BBB opening, despite delivery of a virus and expression of a foreign protein in target neurons. The present study demonstrated that transitory BBB disruption using MRgFUS can be a safe and efficient method for site-specific delivery of viral vectors to the brain, raising the potential for non-invasive focal human gene therapy for neurological disorders.

In one embodiment, the disclosure provides a non-invasive method to deliver a therapeutic or prophylactic transgene to one or more regions of a central nervous system. e.g., brain or spinal cord, in a mammal having a neurocognitive, neurodegenerative, cardiovascular or cerebrovascular disease. The method includes administering to the mammal an amount of a plurality of microbubbles and an amount of a recombinant virus, i.e., a non-oncolytic virus, or a plasmid comprising the transgene and applying focused ultrasound to one or more regions of the central nervous system or the periphery of the mammal in an amount that allows the recombinant virus or the plasmid to cross the blood brain barrier or enter tissue in the periphery. In one embodiment, the focused ultrasound is applied to the striatum, hippocampus or basal forebrain. In one embodiment, the transgene encodes tyrosine hydroxylase, p11, LDL-R or nerve growth factor. In one embodiment, MR imaging is employed before and/or after the focused ultrasound. In one embodiment, frameless navigation is employed. In one embodiment, the transgene is flanked by recombination sites. e.g., lox sites. In one embodiment, Cre is delivered systemically. In one embodiment, Cre is delivered before focused ultrasound. In one embodiment, Cre is delivered after focused ultrasound. In one embodiment, the transgene encodes a therapeutic protein and further encodes Cre which is flanked by lox sites. In one embodiment, a Cre transgene is delivered via a retrograde vector.

Also provided is a non-invasive method to deliver a prophylactic or therapeutic transgene to one or more regions of a brain or spinal cord of a mammal. The method includes administering to a mammal in need thereof, an amount of a population of microbubbles and an amount of a recombinant virus, i.e., a non-oncolytic virus, or a plasmid comprising the transgene and applying to one or more regions of the central nervous system of the mammal focused ultrasound in an amount that provides for delivery of the recombinant virus or the plasmid to the one or more regions of the brain or spinal cord. In one embodiment, the microbubbles comprise a mammalian serum protein. In one embodiment, the mammal is a human. In one embodiment, the method further comprises administering a magnetic resonance imaging (MRI) contrast agent to the mammal. In one embodiment, the microbubbles and the recombinant virus, i.e., a non-oncolytic virus, or the plasmid are concurrently administered. In one embodiment, a composition comprising the microbubbles and the recombinant virus, i.e., a non-oncolytic virus, or the microbubbles and the plasmid is administered, e.g., a composition where the virus is encapsulated in the microbubbles which may be disrupted at certain temperatures, e.g., about 41-43° C., or certain applied energies. In one embodiment, the focused ultrasound is applied after the microbubbles and the recombinant virus, i.e., a non-oncolytic virus, or the plasmid are administered. In one embodiment, the focused ultrasound is applied concurrently with the administration of the microbubbles and the recombinant virus, i.e., a non-oncolytic virus, or the plasmid. In one embodiment, the microbubbles and the recombinant virus, i.e., a non-oncolytic virus, or the plasmid are directly injected. In one embodiment, the microbubbles and the recombinant virus, i.e., a non-oncolytic virus, or the plasmid are systemically administered. In one embodiment, the transgene encodes a protein or a glycoprotein. In one embodiment, the transgene encodes a recombinase. In one embodiment, the transgene is flanked by recombination sites for a recombinase. In one embodiment, the mammal is administered the recombinase. In one embodiment, a retrograde virus, i.e., a non-oncolytic HSV or poliovirus virus, is employed to deliver a recombinase gene. In one embodiment, the transgene is inactivated by subsequent delivery of a recombinase enzyme or a gene encoding a recombinase enzyme which prevents transgene expression. In one embodiment, the transgene is operably linked to a cell type-specific promoter. In one embodiment, a target sequence for a microRNA is inserted into the transgene and wherein the corresponding microRNA is expressed in an organ or tissue region where transgene expression is undesirable. In one embodiment, the transgene encodes a miRNA or siRNA. In one embodiment, the mammal has Parkinson's disease, Alzheimer's disease, depression, or dementia and expression of the transgene prevents, inhibits or treats one or more symptoms or a pathology of Parkinson's disease, Alzheimer's disease, depression, or dementia. In one embodiment, the mammal has a cardiovascular disease or a cerebrovascular disease and expression of the transgene prevents, inhibits or treats one or more symptoms or a pathology of the disease. In one embodiment, the virus comprises adeno-associated virus, adenovirus, lentivirus, or herpes simplex virus, i.e., a non-oncolytic HSV or poliovirus virus. In one embodiment, the virus or the plasmid is encapsulated in or attached to the microbubbles. In one embodiment, the plasmid is encapsulated in or attached to a nanoparticle or a liposome. In one embodiment, the focused ultrasound is applied to the striatum, hippocampus, or basal forebrain.

Further provided is a non-invasive method to inhibit, restrict or prevent expression in specific regions in a brain or spinal cord of a mammal. The method includes administering to a mammal in need thereof, an amount of a population of microbubbles and an amount a recombinant virus, i.e., a non-oncolytic virus, or a plasmid comprising a therapeutic or prophylactic transgene and a target sequence for a microRNA inserted into the transgene, wherein the corresponding microRNA is expressed in one or more regions of the brain or spinal cord where transgene expression is undesirable; and applying to the central nervous system of the mammal focused ultrasound in an amount that provides for delivery of the recombinant virus or the plasmid to the central nervous system. In one embodiment, the virus or the plasmid is in a microparticle or in the microbubbles that is/are disrupted in the ultrasound field or by a specific temperature. In one embodiment, the transgene is flanked by recombination sites for a recombinase. In one embodiment, the mammal is administered the recombinase. In one embodiment, the recombinase comprises Cre, Flp and PhiC31.

### BRIEF DESCRIPTION OF FIGURES

FIG. 1. MRgFUS facilitates AAV-mediated gene delivery to the brain. A) Gd-DTPA-enhanced T1-weighed images collected post sonication showed disruption of BBB and Gd-DTPA extravasation in brain parenchyma (yellow dashed line). B) The brain tissue was harvested 3 weeks post sonication and the colocalization of GFP and NeuN was visualized using immunostaining. C) DAB visualization of GFP transduction from serial sections centered on targeted area (serial sections through the center of the targeted points) (Scale bar=50 μm).

FIG. 2. MRgFUS facilitates stable, long-term GFP transduction. A) High-magnification immunostaining for GFP and NeuN reveals a dominantly neuronal population of GFP transduced cells in the striatum. (Scale bar=50 μm). B) Quantification of striatal GFP transduction is stable over time. GFP positive neurons are expressed as percent of total number of striatal neurons per 20 high power fields per animal (see Methods). C) The MRgFUS-mediated GFP transduction is restricted mainly to neurons.

FIG. 3. Detection of striatal MRgFUS-facilitated AAV-mediated GFP transduction 16 months post sonication. Gd-DTPA-enhanced T1-weighed images collected post sonication showed disruption of BBB and Gd-DTPA extravasation in brain parenchyma (yellow dashed line). Histological analysis of the brain harvested 16 months post sonication showed GFP transduction in mostly neurons. (Scale bar=50 μm).

FIG. 4. MRgFUS-facilitated AAV-mediated gene delivery in peripheral organs is present short-term but not long-term. Analysis of high-power immunofluorescence images of tissue collected from animals with MRgFUS with AAV1/2.GFP and animals with AAV1/2.GFP stereotactically administered in striatum shows no long-term GFP transgene expression in liver, heart and lungs. (Scale bar=100 μm).

FIG. 5. No evidence of long-term inflammatory response induced by unilateral striatal MRgFUS-mediated gene transfer to the brain. Both Iba1 (microglia marker) and GFAP (astrocytic marker) returned to baseline levels by week 2, suggesting that local inflammatory response is transitory. (Scale bar=50 μm).

FIG. 6. Experimental design. Animals underwent sonication, simultaneously with administration of Optison microspheres (MB), AAV.1/2.GFP and contrast agent Magnevist (Gd-DTPA). T2-weighted and T1-weighted MRI images were used for detecting the target and confirming Gd-DTPA extravasation, respectively. The brains and organs were harvested and processed for histological analysis at different time points post-sonication.

FIG. 7. Tissue integrity three weeks post-sonication. Immunostaining for NeuN (neuronal marker) and GFP (expressed transgene) showed no neuronal death post-sonication. Hematoxylin and eosin staining confirmed that no tissue damage was detected at three weeks post MRgFUS.

FIG. 8. Stable GFP expression throughout the brain. DAB visualization of GFP transduction from serial sections centered on targeted area (serial sections through the center of the targeted points).

FIG. 9. MRgFUS facilitates GFP transduction up to 16 months post-sonication. A) The MRgFUS-mediated GFP transduction is restricted mainly to neurons. B) Quantification of GFP transduced cells reveals persistent GFP transduction at 16 months post-sonication. GFP positive neurons are expressed as percent of total number of neurons per 20 high power fields per animal (see Methods).

FIG. 10. GFP transduction of the cortex on the FUS trajectory follows the pattern of the targeted region. A) Quantification of cortical GFP transduction is stable over time. GFP positive neurons are expressed as percent of total number of cortical neurons per 20 high power fields per animal (see Methods). B) The MRgFUS-mediated GFP transduction is restricted mainly to neurons.

FIG. 11. Total neurons in the sonicated area and matching contralateral non-sonicated brain area for the striatum (top) and cortex (bottom) at 2 weeks, 2 months and 6 months following focused ultrasound mediated gene delivery. Range of total neuronal counts is similar across time and between the sonicated and non-sonicated hemispheres.

FIG. 12. No evidence of long-term inflammatory response in the cortex located on the trajectory of the focused ultrasound. Both Iba1 (microglia marker) and GFAP (astrocytic marker) returned to baseline levels by week 2, suggesting that local inflammatory response in the cortex is transitory. (Scale bar=50 μm).

FIG. 13. Focused ultrasound induced transitory inflammatory response limited to the sonicated striatal area. Immunohistological analysis revealed that both Iba1 (microglia marker) and GFAP (astrocytic marker) are increased at 3 hrs, 24 hrs, and 48 hrs post-sonication, in the sonicated striatum. (Scale bar=100 μm).

FIG. 14. Focused ultrasound induced transient inflammatory response in the cortex located on the trajectory of focused ultrasound. Immunohistological analysis revealed that both Iba1 (microglia marker) and GFAP (astrocyte marker) are increased at 3 hrs, 24 hrs, and 48 hrs post-sonication. (Scale bar=100 μm).

### DETAILED DESCRIPTION

Gene therapy has long held promise as a potentially groundbreaking method for improving a variety of complex disorders. The brain has been a major focus of translational gene therapy research, with several human clinical trials showing safety and evidence of efficacy for Parkinson's disease (Kaplitt et al, 2007; LeWitt et al., 2011; Marks et al., 2010; Marks et al., 2008), Alzheimer's disease (Rafii et al., 2014; Tuszynski et al., 2005; Tuszynski et al., 2015) and a variety of neurogenetic disorders. Due to the presence of the blood-brain-barrier (BBB) and its highly selective permeability (Abbott et al., 2010; McCaffrey & Davis 2012), the only current means for efficient delivery of viral vectors to specific regions in the human brain has been through invasive direct injection. This not only carries the attendant risks of invasive surgery, but efficient distribution of gene therapy agents throughout a target area can be difficult to confirm with traditional infusion methods. Newer approaches have been tested, which permit monitoring of contrast spread during infusion as a surrogate for viral vector distribution utilizing specialized catheter systems with intraoperative magnetic resonance imaging (MRI) methodology (Fiandaca et al., 2009; Salegio et al., 2012).

In order to reduce surgical risks and avoid complexities of direct infusion, non-invasive approaches have been explored to permit intravenous delivery of viral vector into the brain. Use of an osmotic agent such as mannitol has long been known to transiently open the BBB, permitting delivery of a variety of agents to the brain, including viral vectors (Carty et al., 2010; Neuwelt et al., 1985; Neuwelt et al., 1979; Schuster et al., 2014). However, systemic administration of mannitol induces widespread opening of the BBB, precluding target specific gene expression. Elective intra-arterial delivery of BBB disruption agents could provide more targeting gene delivery and cover larger brain areas, but variability in vascular supply of various important deep brain structures creates challenges for reproducible delivery between individuals (Foley et al., 2014).

An alternative to chemical delivery is mechanical disruption of the BBB. One approach is MR guided focused ultrasound (MRgFUS) (Hynynen et al., 2007; Hynynen et al., 2001; McDannold et al., 2005). This involves focused delivery of ultrasound to a target region, and high frequency MRgFUS has been used in human patients to create targeted brain lesions to treat essential tremor and pain (Elias et al., 2013; Elias et al., 2016; Jeanmonod et al., 2012). Use of MRgFUS at lower frequency, in combination with microbubble-mediated cavitation, has been shown to focally open the BBB to facilitate transfer of drugs (Treat et al., 2012), antibodies (Jordao et al., 2013), and nanoparticles (Nance et al., 2014) from the blood stream to the brain parenchyma. This has also been utilized for non-invasive gene delivery to the rodent brain (Alonso et al., 2013; Hsu et al., 2013; Huang et al., 2012; Thevenot et al., 2012; Wang et al., 2015). These reports have shown successful transfer of adeno-associated virus (AAV) vectors from the blood stream to the brain following MRgFUS-mediated BBB opening with variable efficiencies and with transgene expression evaluated for relatively short periods following delivery (Alonso et al., 2013; Hsu et al., 2013; Thevenot et al., 2012; Wang et al., 2015). Since the goal of gene delivery is long-term neuronal modification, long-term expression following MRgFUS-mediated gene delivery remains to be confirmed. This is particularly important since potential immune-mediated loss of gene expression or transduced cells, due to exposure of the brain to the immune system following BBB disruption, might not fully manifest until later time points, as has been observed in some gene transfer studies outside of the brain (Bell et al., 2011; Breous et al., 2011; Manno et al., 2006; Mingozzi et al., 2011; Wang et al., 2005). Furthermore, the potential for provoking inflammation in brain parenchyma following the application of focused ultrasound has only been examined up to 2 weeks following either MRgFUS BBB disruption alone (Jordao et al., 2013; Kovacs et al., 2017) or following delivery of AAV vectors (Thevenot et al., 2012; Wang et al., 2017). The potential consequences of long-term brain exposure to a potential immunogenic viral vector following BBB disruption remain unknown.

As described herein below, MRgFUS-mediated BBB disruption can lead to efficient delivery and wide distribution of AAV vectors to the intended brain target in rodents. It was demonstrated that gene expression is stable over extended periods of time (6 months to 16 months), comparable to what has been historically observed with direct infusion. Finally, while a mild initial inflammatory response was observed for the first two days following BBB disruption, no evidence of inflammation over the long-term was observed and no evidence of behavioral or histological toxicity was noted at any time point.

### EXEMPLARY EMBODIMENTS

In one embodiment, the disclosure provides a method of delivering genes to the brain or spinal cord. The method includes transiently disrupting the blood-brain barrier in a targeted brain region of a mammal using focused ultrasound and administering, e.g., systemic delivery of, a genetic vector. In one embodiment, the ultrasound field is targeted to a brain region using MRI guidance. In one embodiment, the method further comprises administering microbubbles, e.g., intravenously, in an amount to facilitate transient opening of the blood-brain barrier. In one embodiment, the genetic vector is delivered intravenously or intra-arterially. In one embodiment, the genetic vector comprises an adeno-associated virus, adenovirus, lentivirus, herpes simplex virus, i.e., a non-oncolytic virus, or a plasmid. In one embodiment, the genetic vector is encapsulated in or attached to the microbubbles used for blood-brain barrier disruption. In one embodiment, the method does not employ the use of an osmotic agent.

Also provided is a method of restricting expression to a target organ, tissue region or cell type by preventing gene expression in undesired targets. In one embodiment, the target organ is brain or spinal cord region. In one embodiment,

the vector is prevented or inhibited from delivering genes to cells outside of the ultrasound field. In one embodiment, a target sequence for a microRNA is inserted into the gene to be expressed in the brain or spinal cord wherein the corresponding microRNA is expressed in an organ or tissue region where transgene expression is undesirable. In one embodiment, the genetic vector is encapsulated in a microparticle that is disrupted in the ultrasound field, permitting gene delivery to the tissue in that field. In one embodiment, the transgene is inactivated by subsequent delivery of a recombinase enzyme or gene encoding a recombinase enzyme which prevents transgene expression. In one embodiment, the recombinase system comprises Cre, Flp or PhiC31.

The invention thus provides materials and methods useful for ultrasound-mediated non-invasive delivery of gene therapy agents to the central nervous system. In one example, MR guided focused ultrasound is used in combination with microbubbles to facilitate gene delivery to a particular region of the brain. Microbubbles are injected intravenously immediately before or during the procedure to delivery ultrasound to one or more central nervous system target(s). Opening of the BBB is then assessed by MRI evidence of extravasation of a contrast agent such as gadolinium (GAD) into the brain following intravenous injection. In another example, the ultrasound is delivered without MR guidance to a brain target using either frameless navigation and targeting of the ultrasound source to a planned surface area of the scalp, or the ultrasound source is inserted into the skull aimed at a planned trajectory to allow targeting of the ultrasound to a desired volume of brain tissue. In one example, the viral agent may then be injected either simultaneously with the GAD contrast agent or up to 24 hours later. In one embodiment, the gene therapy agent is an adeno-associated virus (AAV) vector. e.g., a recombinant AAV serotype 1/2 vector, delivered intravenously. In one embodiment, the gene therapy vector, e.g., a viral vector such as a rAAV, can then deliver the gene for tyrosine hydroxylase (TH) to the striatum to increase dopamine production and improve symptoms of Parkinson's disease. In one embodiment, the gene therapy vector, e.g., a viral vectors including but not limited to a rAAV, excluding an oncolytic virus, can be used to deliver the gene for p11 to the striatum to improve symptoms of depression. In one embodiment, the gene therapy vector can also be used to deliver the gene for the low density lipoprotein receptor (LDL-R) to the hippocampus to reduce pathology of Alzheimer's disease, such as amyloid deposition and hyperphosphorylated Tau. In one embodiment, the gene therapy vector can also be used to deliver a microRNA (miRNA) or small interfering RNA hairpin (shRNA), for example, directed against IDOL, to prevent expression of the LDL-R processing enzyme, thereby increasing endogenous or exogenous LDI-R. In one embodiment, the gene therapy vector can also be used to delivery LDL-R cDNA and the IDOL shRNA or miRNA in a single vector. In this example, the gene therapy vector(s) expressing LDL-R and/or IDOL are not prevented from transducing systemic organs such as liver, since transduction of the liver and overexpression of the LDL-R leads to reduction in blood LDL levels, which should be additionally beneficial for both Alzheimer's disease and for cardiovascular and cerebrovascular diseases. In one embodiment, the gene therapy vectors can also be used to deliver the gene for nerve growth factor (NGF) to the basal forebrain to prevent degeneration of cholinergic neurons of that region and reduce progression of Alzheimer's disease or other dementing illnesses. In one embodiment, the gene therapy vector is a lentivirus vector. In one embodiment, the gene therapy vector is an adeno-associated vector. In still another example, the gene therapy agent is a plasmid, e.g., encapsulated in a nanoparticle or liposome.

One potential limitation of opening the BBB is the possibility of causing inflammation due to exposure of the brain to immune cells in the bloodstream. This inflammation can cause toxicity to the brain, and can limit long-term expression of therapeutic genes from otherwise stable gene therapy agents due to immune system attacks on transduced cells which either destroy the cells or induce destruction of the transgene without cell death. This invention provides for a method of gene delivery using focused ultrasound to transiently disrupt the BBB such that no long-term inflammation occurs, thereby preventing loss of brain cells and facilitating long-term expression of gene products for neurological or psychiatric disorders.

In another example, a therapeutic gene is delivered using a gene therapy agent and focused ultrasound over several sessions in order to optimize coverage of a particular region or to target multiple regions independently. In this example, vectors are delivered in subsequent sessions at least 24 hours after the previous session, in order to re-establish an effective blood brain barrier within the previously targeted area. Since exposure to the immune system can limit further transduction of the same vector in subsequent sessions, in another example, the therapeutic expression cassette is delivered in subsequent sessions using a different strain of viral vector or using an entirely different gene therapy agent than the previous session.

In another embodiment, the gene therapy agent is restricted from expressing a functional therapeutic protein outside of the desired central nervous system target in the field of the ultrasound. This may be preferable when the therapeutic gene in one cell type within the brain region targeted by the ultrasound field might have adverse effects when expressed in other cell types within that region. This may also be preferable when systemic administration of a gene therapy agent could lead to transduction of peripheral organs outside of the CNS, and when such transduction could lead to toxicity due to production of a functional gene product within an unintended organ. In one example, the therapeutic gene or a portion of the expression cassette is flanked by lox sites (“floxed”) such that recombination will delete the functional expressing unit and prevent further gene expression. After delivery of this agent to the desired central nervous system target using focused ultrasound, at least 24 hours is allowed to pass to permit closure of the BBB, whereupon a second gene therapy agent expressing the Cre recombinase enzyme is given systemically. This cannot enter the central nervous system due to closure of the BBB, leading to transduction exclusively within peripheral organs, whereup Cre expression leads to recombination and deletion of the expression cassette within peripheral organs. In another example, the Cre-expressing vector is also flanked by lox sites such that Cre expression from this vector not only deletes the original transgene, but also deletes the Cre expression cassette itself, thereby preventing further Cre expression. In another example, a Cre vector is delivered first, prior to opening of the BBB, and then at a future timepoint beyond 24 hours, the therapeutic vector sensitive to Cre is delivered and BBB opening is performed, thereby causing more immediate recombination and inhibition of expression in the periphery upon delivery of the therapeutic agent. In still another example, the viral gene therapy agent or plasmid DNA are attached to or encapsulated within microbubbles used for BBB disruption, and then released in the field of the ultrasound. In still another example, a recognition sequence for an endogenous miRNA expressed within an undesirable organ or cell type is inserted into the cDNA for the therapeutic transgene expressed from the gene therapy vector, thereby preventing expression of a functional therapeutic protein within undesirable organs or cell types which express the cognate miRNA.

In one example, sequences for miR122, which is a liver specific miRNA, are employed in a gene therapy vector to prevent expression in the liver, e.g., to more efficiently target the expression of the vector to the brain after MRgFUS. In another example, miRNAs that are specific for cells in the brain, such as neurons, astrocytes, oligodendrocytes and/or microglia, may be employed. In one embodiment, miR124 is employed as it is expressed in neurons and not in other cells types, and since astrocytes and oligodendrocytes are what give rise to brain tumors, the use of a vector having a miR124 sequence would further prevent viral replication in neurons, and adding a miR124 target sequence to the non-coding region of the cDNA for the transgene would prevent expression in neurons and would permit expression only in cells that comprise the tumor. In another example, microRNAs expressed mostly in D1 dopamine receptor neurons in the putamen and not in the D2 neurons could be employed in diseases such as Parkinson's disease or Huntington's disease. Exemplary miRNAs for regulating expression in the brain include but are not limited to those for neurons: miR-7b, miR-124, miR-124, miR-127, miR-128, miR-129, miR-129, miR-132, miR-135b, miR-136, miR-136, miR-137, miR-139-5p, miR-154, miR-184, miR-188, miR-204, miR-299, miR-300-3p, miR-300-5p, miR-323, miR-329, miR-337, miR-335, miR-341, miR-369-3p, miR-369-5p, miR-376a, miR-376a, miR-376b-3p, miR-376b-5p, miR-376c, miR-377, miR-379, miR-379, miR-382, miR-382, miR-409-5p, miR410, miR411, miR-431, miR-433, miR-434, miR-451, miR-466b, miR-485, miR-495, miR-539, miR-541, miR-543, miR-551b, miR-758, or miR-873; for astrocytes: miR-21, miR-31, miR-34b, miR-34c, miR-135a, miR-143, miR-146a, miR-193, miR-210, miR-221, miR-222, miR-223, or miR-449a; and for oligodendrocytes: miR-17-3p, miR-20a, miR-20b-5p, miR-219-2-3p, miR-219-5p, miR-322, miR-338, miR-338, miR-346, miR-351, miR-450a, miR-503, or miR-542-3p.

In another example, an inactive floxed therapeutic vector, which is activated upon Cre expression through deletion of a disruptive DNA sequence, is delivered to a target brain region, and more than 24 hours later, a retrograde vector (such as retroAAV or canine adenovirus but not an oncolytic virus) expressing Cre is delivered to a brain region that synapses with axons from a subset of neurons emanating from the original target region. This leads to uptake of the retrograde vector into the axons and transport back for Cre expression only within the subset of neurons that project to the second brain region targeted in the second session, resulting in expression of the therapeutic gene only within the desired subset of neurons within the original targeted brain region. In another example, a cell-type specific promoter is used to drive expression of the therapeutic gene within a brain target, thereby restricting expression to one or more subsets of cell types within the target tissue following ultrasound-mediated gene delivery into the brain.

### Exemplary Viral Gene Therapy Vectors

### Lentiviral Vectors

Lentiviruses are able to infect both mitotic and post-mitotic cells, such as glia and neurons, respectively, and translocate across the nuclear membrane and stably integrate into chromosomes, allowing them to mediate long-term gene expression while producing only a minimal immune response. Lentiviral vectors can accommodate up to 16 kb proviral length; however, the maximal packaging size of lentiviral vectors is estimated to be approximately 11 kb. Replication-deficient, self-inactivating vectors lead to long-term expression of transgenes with minimal immune response and inflammation. In addition to HIV-1-based lentiviral vectors, equine infectious anemia virus (EIAV), simian immunodeficiency virus (SIV) and feline immunodeficiency virus (FIV), may be employed for gene delivery to the CNS.

### Adeno-Associated Viral (AAV) Vectors

Many different serotypes of AA many be employed to deliver genes, e.g., any one of AAV 1-9, AAVrh10. AAV vectors can transduce both dividing and non-dividing cells, can offer stable long-term expression, and can be generated at high titers. AAV vectors may be generated via a “helper-free” system which avoids the helper virus infection. Self-complementary AAV (scAAV) vectors have been developed, scAAV vectors may achieve a better foreign gene transduction than ssAAV. AAV2 has a neuronal tropism. Other serotypes may be employed for gene delivery to the CNS including but not limited to AAV1, AAV4, AAV5, AAV6, AAV8, AAV9, and AAV rh10.

### Exemplary Transgenes and Disorders to be Treated

The methods described herein may be employed to prevent, inhibit or treat one or more neurological disorders or symptoms thereof including but not limited to Alzheimer's disease, Parkinson's disease, depression, epilepsy, or dementia or other dementing diseases

The methods described herein may be employed to prevent, inhibit or treat one or more cerebrovascular disorders or symptoms thereof including but not limited to cerebral stroke or subarachnoid hemorrhage

The methods described herein may be employed to prevent, inhibit or treat one or more brain cancers including but not limited to acoustic neuroma, astrocytoma, glioblastoma (GBM), chordoma, CNS lymphoma, craniopharyngioma, medulloblastoma, meningioma, metastatic brain tumors, oligodendroglioma, pituitary tumors, primitive neuroectodermal tumors (PNET), schwannoma, brain stem clioma, craniopharyngioma, ependymoma, juvenile pilocytic astrocytoma (JPA), medulloblastoma, optic nerve glioma, pineal tumor, or rhabdoid tumor.

### Exemplary Microbubbles

Microbubbles are smaller than one hundredth of a millimeter in diameter, but larger than one micrometer. In one embodiment, the microbubbles have an average diameter of about 1 to 10 μm. In one embodiment, the microbubbles have an average diameter of about 2 to 8 μm. In one embodiment, the microbubbles have an average diameter of about 10 to 100 μm. In one embodiment, the microbubbles have an average diameter of about 10 to 20 μm. In one embodiment, the microbubbles have an average diameter of about 20 to 80 μm. They include a shell that encapsulates a material, e.g., a shell that is gas-filled, e.g. air or perfluorocarbon. The shell may be formed of a lipid or a protein. In one embodiment, microbubbles are formed of a human serum protein such as serum albumin which encapsulates a gas such as perfluoropropane.

### Exemplary Conditions

In one embodiment, the acoustic pressure may be from about 0.2 to about 2.5 Mpa, e.g., about 0.6, 1.2, or 1.8 MPa. In one embodiment, one or more sonication points may be employed, e.g., 1, 2, 3, 4, 5, 6, 7 or more sonication points. In one embodiment, sonication times may be from about 5 seconds to about 400 seconds, e.g. from 120 seconds to 200 seconds. In one embodiment, the transducer is a spherically focused transducer (e.g., 7-cm diameter, F #: 0.8, FUS Instruments, Canada) with a fundamental frequency of 1.145 MHz. In one embodiment, more than one transducer is employed.

In one embodiment, for AAV, an acoustic pressure amplitude of about 1 to about 3, e.g., about 1.9, MPa is employed. In one embodiment, an in situ acoustic pressure of about 0.5 to about 2.0, e.g., about 0.97, MPa is attained. In one embodiment, a burst length of about 5 msec to about 20 msec, e.g., about 10 msec, is employed. In one embodiment a pulse-repetition Frequency of about 0.5 to about 2.5 Hz, e.g., about 1 Hz, is employed. In one embodiment, the period is about 500 to about 1500 msec, e.g., about 1000 msec. In one embodiment, the total sonication time is about 30 seconds to about 240 seconds, e.g., 200 seconds.

The invention will be further described by the following non-limiting examples.

### Example 1

### Materials and Methods

### Animal Preparation/Experimental Design

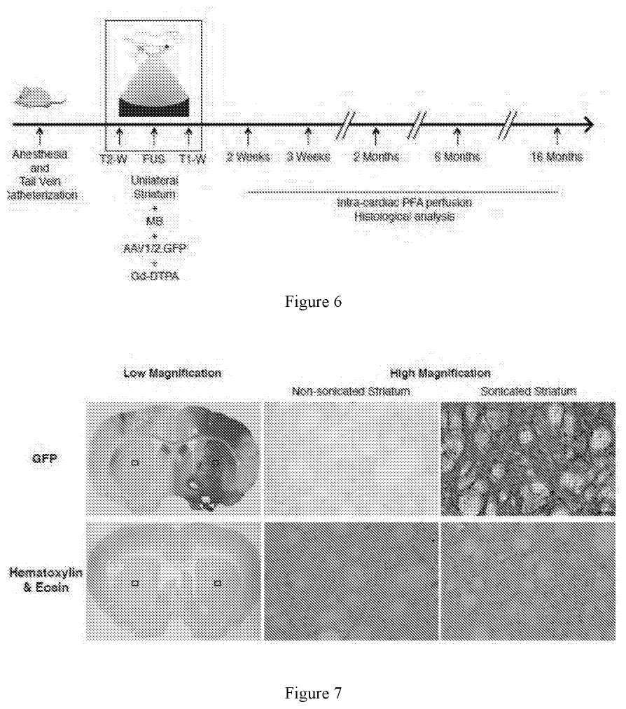

All animal procedures were approved by the Institutional Animal Care and Use Committee of Weill Cornell Medical College and followed National Institutes of Health guidelines. Ten-week old Sprague-Dawley male rats (250-300 g). Charles River Laboratories, Wilmington, Mass.) were used in all studies. Four rats were used to assess the efficiency and safety of unilateral MRgFUS-mediated AAV.GFP delivery to the striatum at three weeks post sonication. Another group of fifteen rats underwent a similar procedure but were sacrificed at different time points: 2 weeks (n=3), 2 months (n=4), 6 months (n=6) and 16 months (n=2). The brains and several organs (liver, lung, and heart) were then harvested and processed for histological analysis (FIG. 6).

### Recombinant AAV Vectors

AAV1/2 hybrid vector stocks, encoding the reporter gene green fluorescent protein (GFP) under the control of CAG promoter, were prepared by packaging the plasmids into AAV particles containing capsid proteins for both AAV1 and AAV2 using a helper-free plasmid transfection system that we have described previously (Kaplitt et al., 1994; Morgenstern et al., 2011). Vectors were purified using heparin affinity chromatography and dialyzed against PBS. rAAV titers were determined by quantitative PCR using CMV-enhancer-specific primers and adjusted to 10⁹ genomic particles per μL.

### MRI-Guided Focused Ultrasound and Viral Vector Delivery

Animals were anesthetized using a Ketamine (90 mg/kg) and Xylazine (4 mg/kg) cocktail. A 22 g IV catheter (BD InsyteAutoguard) was inserted into the lateral tail vein for substance administration during experiments. After scalp shaving, animals were secured in a supine position on the FUS system and the head was coupled with the degassed water tank holding the transducer. The spherically-focused transducer (7 cm diameter, f #=0.8) was driven by a computer-controlled function generator (33220A Agilent Function/Arbitrary 20 MHz waveform generator; Agilent Technologies, CA) and a 43 db RF power amplifier (FUS Instruments, Inc).

Before sonication, an MRI was performed with a 3.0T GE scanner, using a 4×7 cm RF surface coil. T2-weighted axial images, 10 slices, perpendicular to the direction of the ultrasound beam propagation, were acquired before sonication to calculate the coordinates of the target. The transducer was then moved to the desired position using a motorized three-axis positioning system (FUS Instruments. Inc). The striatum was sonicated in four points, 1.5 mm apart. Assuming a 49% loss of ultrasound power due to attenuation through the rat skull (Treat et al., 2007), an estimated in situ rarefactional pressure of 0.97 MPa was applied at the sonication points, with a 1 Hz pulse repetition frequency, 10 ms burst length, and 200 s total sonication time. The cocktail of rAAV and Optison microspheres (Perflutren Protein-Type A microspheres, mean size 3-4.5 μm, 0.4×10⁸-0.64×10⁸/kg, GE Healthcare Life Sciences) was administered simultaneously with sonication through the tail vein catheter, followed by MRI contrast agent Magnevist (gadopentetate dimeglumine, Gd-DTPA, 0.4 ml/kg; Bayer, Germany). T1-weighted images, 7 slices, were collected at the conclusion of sonication to monitor the degree of the BBB opening based upon contrast extravasation. The slice thickness was 0.8 mm, with a spacing of 0.2 mm.

### Immunohistochemistry and Histology

Rats were deeply anesthetized with sodium pentobarbital (150 mg/kg) and transcardially perfused with ice-cold 0.1 M heparinized PBS (pH 7.4) followed by 4% (wt/vol) buffered paraformaldehyde (PFA) solution. The brains, liver, heart and lungs were removed, post-fixed in the same fixative solution for 24 hours, and subsequently immersed in 30% (wt/vol) sucrose cryoprotective solution at 4° C. The brains were frozen and sectioned serially (six series per brain) into 40 μm thick sections in the coronal plane on an AO Spencer 860 sliding microtome. The organs were embedded in agar solution and 40 μm thick sections were cut on a Leica VT1200 vibratome for histological analysis.

To determine the extent of the area transduced by GFP, an entire series of brain coronal sections per each animal was rinsed in Tris-buffered saline with 0.1% Triton (TBST). Following the quenching of endogenous peroxides with a 0.3% solution of hydrogen peroxide in TBST, sections were incubated in blocking solution (3% BSA and 2% goat serum in TBST) for 1 hour at room temperature and then for 24 hours at 4° C. with a rabbit polyclonal anti-GFP antibody (Abcam, ab290, 1:4000). The following day, sections were rinsed in TBST before a 1 hour incubation with biotinylated secondary antibodies. Following several washes, sections were incubated with Vectastain Elite ABC kit (1:500) in TBST for 1 hour. Staining was visualized using a 3,3′-diaminobenzidine (DAB) Peroxidase Substrate solution.

Identification of the neurons transduced by GFP was performed using an immunofluorescence protocol. Sections were incubated in blocking solution (3% BSA and 2% goat serum in TBST) for 1 hour at room temperature and then for 24 hours at 4° C. with a rabbit polyclonal anti-GFP (Abcam, ab290, 1:4000), and mouse monoclonal anti-NeuN (neuronal marker beta-tubulin III; Abcam. AB104224, 1:1000) antibodies. The following day, sections were rinsed and incubated in goat anti-rabbit Alexa Fluor 488 and goat anti-mouse Alexa Fluor 594 conjugated secondary antibodies (Life Technologies), and nuclei were stained with DAPI (Invitrogen, 1:10,000). Hematoxylin and eosin staining was used to evaluate intact cells and tissue integrity.

For detection of inflammatory markers, sections were incubated with mouse monoclonal anti-Iba1 (Millipore. MABN92, 1:500) and rabbit monoclonal anti-GFAP (Abcam, ab7260, 1:1000) antibodies and the staining was visualized with goat anti-mouse Alexa Flour 488 and goat anti-rabbit Alexa Fluor 594 conjugated secondary antibodies (Life Technologies). The nuclei were stained with DAPI (Invitrogen, 1:10,000).

### Image Analysis/Quantitative Analysis of GFP-Positive Area and Statistical Analysis

Quantitative analysis of the GFP transduced striatum was performed using Image J Fiji software. Z-stacks from four sections per animal were collected at 10× magnification using an Olympus BX61 upright microscope, and cells were quantified from twenty random fields taken from a pre-defined region-of-interest within the sonicated striatum (100×100×40 μm depth). The region-of-interest was kept constant between animals to permit between animal comparison. To determine the proportion of GFP transduced neuronal and non-neuronal cells, colocalization of GFP and NeuN was analyzed using IF microscopy. Neuronal cell transduction rate was calculated by expressing GFP positive neuronal cells as percent of the total number of neurons in the analyzed area. To quantify the distribution of neuronal and non-neuronal cells among GFP positive cells, both GFP positive neuronal cells and GFP positive non-neuronal cells were expressed as percent of total GFP transduced cells.

### Statistics

Two-tailed t-test was employed for statistical comparison of all groups. All data are expressed as the mean value with Standard Error of the Mean (SEM). When the p value was less than 0.05, the difference was considered to be statistically significant.

### Results

### MRgFUS-Facilitated AAV/2-Mediated GFP Gene Transduction of Rat Striatum

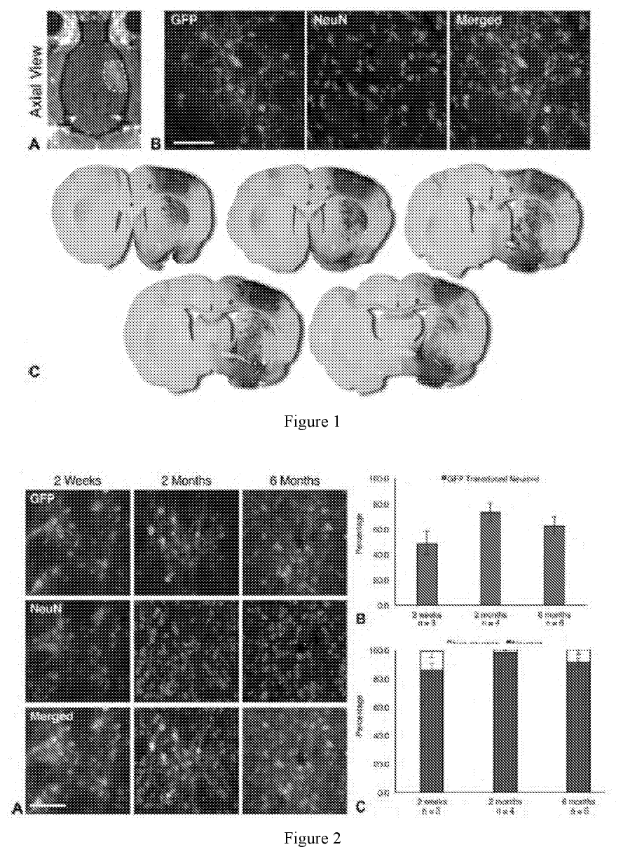

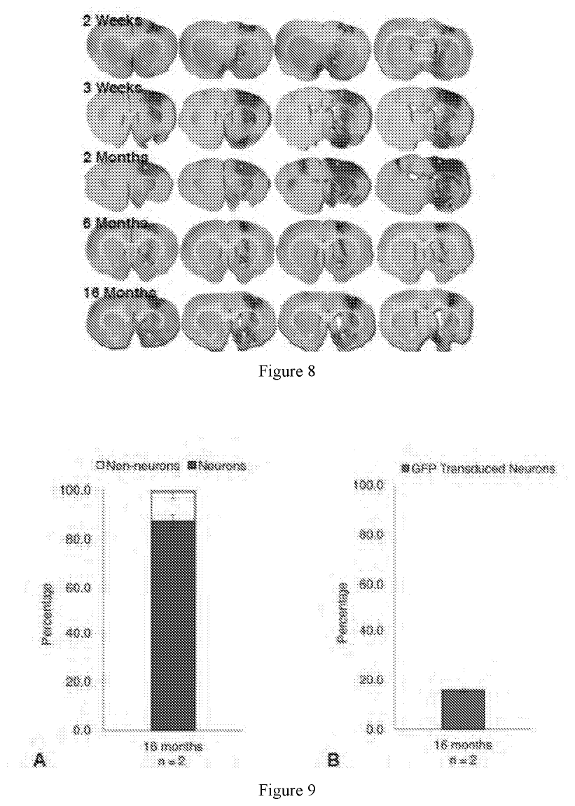

To first evaluate the efficiency of the present system to safely and transiently disrupt the BBB in the striatum, three rats underwent unilateral striatal MRI guided four-point sonication. A post-sonication T1-weighted MRI demonstrated local extravasation of Gd-DTPA into the local brain tissue confirmed BBB disruption (FIG. 1A). Gd-DTPA extravasation overlapped the striatal area selected on the T2-weighted pre-sonication MRI scan. Three weeks later immunohistological analysis confirmed GFP transgene expression in the sonicated striatum, in both neuronal and non-neuronal cells, while no signal was detected on the contralateral side of the brain (FIGS. 1B-C and 7). DAB chromogenic staining of GFP transgene in a series of sections showed an extensive and efficient antero-posterior and dorso-medial GFP transduction of the targeted striatum within the volume of tissue subject to sonication (FIG. 1C). In addition, hematoxylin and eosin staining revealed no evidence of tissue damage in the sonicated area (FIGS. 7-8).

### MRgFUS-Facilitated AAV1/2-Mediated GFP Expression is Efficient and Stable Over an Extended Period of Time

Although long-term stability of AAV-mediated gene expression has been well established in the brain following direct infusion, factors such as exposure to the immune system following BBB disruption could influence longevity of gene expression and survival of transduced neurons. GFP immunostaining with DAB-Peroxidase substrate performed in rat tissue collected six months post sonication revealed long term expression of GFP transgene through extended brain parenchyma.

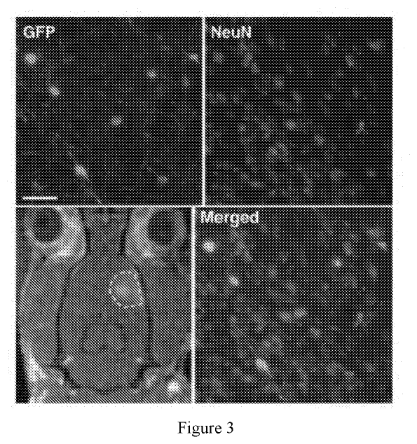

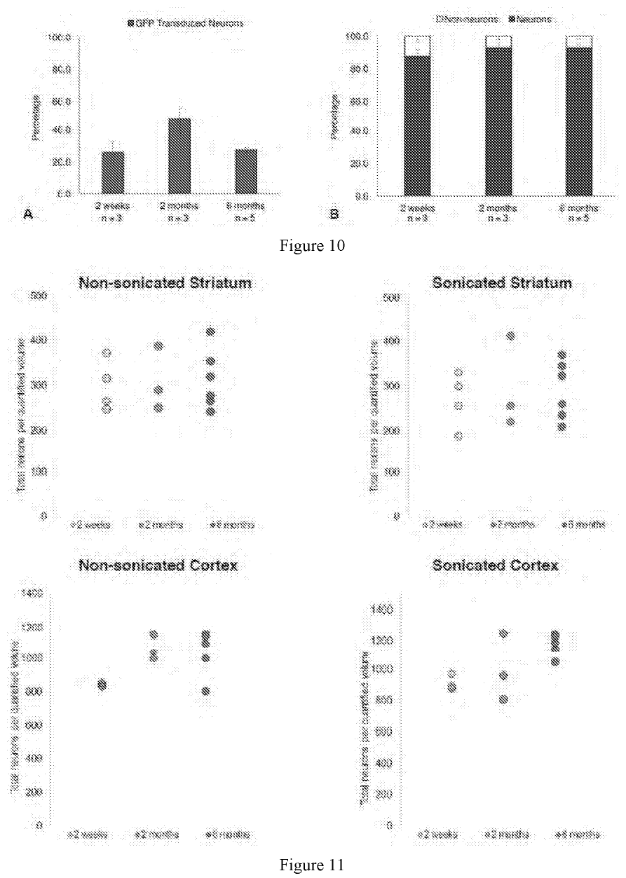

Triple immunolabeling for the GFP transgene with NeuN and DAPI allowed quantification of transduced neurons and non-neuronal cells from 2 weeks to 6 months following MRgFUS-facilitated AAV1/2.GFP delivery (FIG. 2). Quantification of GFP positive neurons from the total number of neurons revealed transduction of between 50% and 74% of neurons within the analyzed region. A lower rate of transduction was observed at 2 weeks (50%), with higher transduction rates noted at 2 months (74%) and 6 months (63%) (FIG. 2B). This could be consistent with the known pattern of AAV-mediated transduction, which generally increases over several weeks following delivery, but the relatively small number of animals in the 2 weeks group could also have influenced this variation. Analysis of the type of GFP transduced cells revealed that neurons represented 86% to 98% of total GFP expressing cells (FIG. 2C), again consistent with previous experience with direct infusion of this particular AAV serotype. Immunohistological analysis of GFP-NeuN colocalization of two animals that were sacrificed at 16 months post facilitated AAV-mediated GFP delivery revealed a similar prevalence of GFP neurons in the transduced striatum (FIGS. 3 and 9). These two subjects were not part of the cohort of animals included in the time course study, but had been treated much earlier during the pilot period, and therefore it is difficult to directly compare cell numbers in these two animals compared with the rest of the time course cohort. Nonetheless, this data shows that there is demonstrable ongoing expression 16 months after MRgFUS-mediated gene delivery. While the focus of our transducer was on the striatum, the nature of the conical ultrasound beam from the single curved transducer resulted in considerable BBB disruption in the overlying cortex. Transduction of the overlying cortex was similar to what we observed with the striatum, suggesting that MRgFUS is capable of efficient and stable gene delivery to different brain regions (FIG. 10). Finally, neuronal quantification revealed no evidence of neuronal loss over time in either the sonicated striatum or cortex and no difference in these regions compared with the corresponding non-sonicated hemisphere (FIG. 11).

### Peripheral MRgFUS-Facilitated GFP Gene Transduction is Transient.

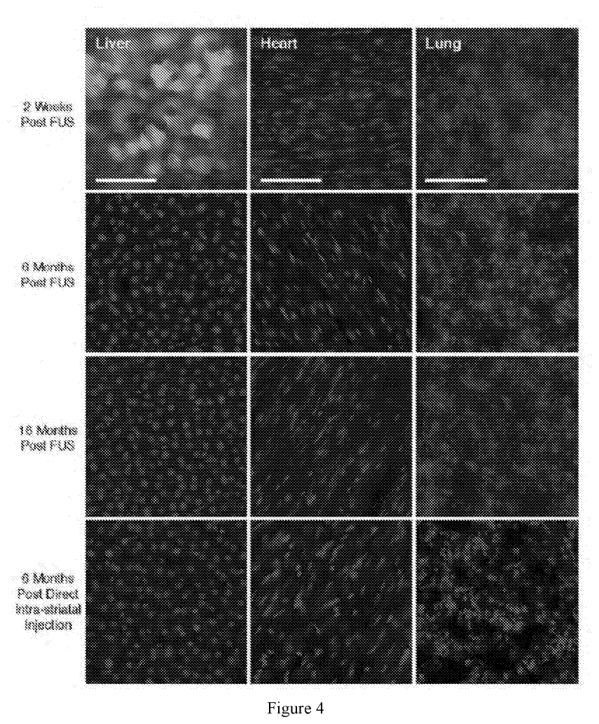

In order to non-invasively deliver AAV into the brain via MRgFUS, vectors were infused intravenously, which could also result in transduction of peripheral organs depending upon the tropism of the serotype utilized. To determine the extent of peripheral gene expression, the heart, lung and liver were harvested at the different time points following unilateral striatal MRgFUS and immunostained for GFP expression. While GFP was detected in the liver 2 weeks post sonication, no signal was detected at later time points (6 months and 16 months), consistent with a likely immune mediated loss of gene expression as observed in other studies of foreign transgenes in this organ (Bell et al., 2011; Manno et al., 2006). Heart and lungs did not test positive for GFP at any time point (FIG. 4). As a negative control, these same organs were harvested from animals in which AAV1/2.GFP was delivered by direct infusion into striatum, with no evidence of gene expression in any organ.

### MRgFUS Induces a Transitory Local Inflammatory Response.

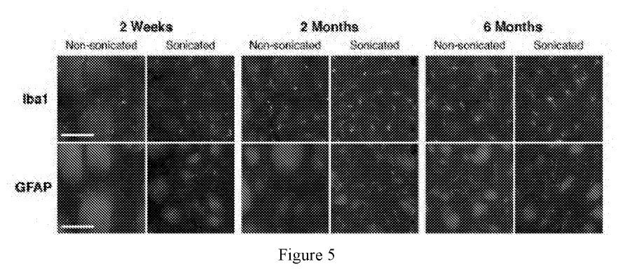

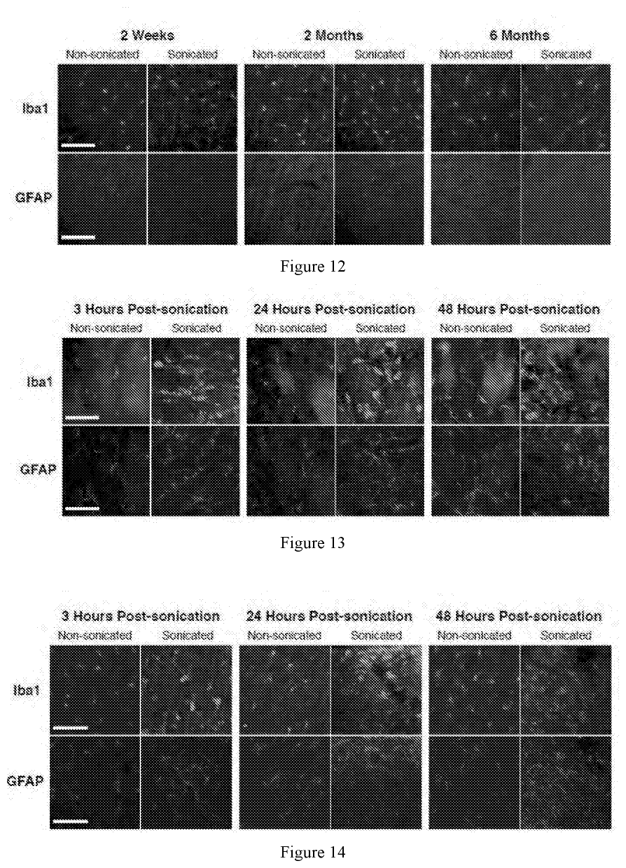

The opening of the BBB, even when transient, could permit passage of various components from the blood stream into brain parenchyma. Previous studies have reported evidence of inflammation in the targeted brain soon after MRgFUS. No astrocytosis or microgliosis was detected in both sonicated and non sonicated striatum at 3 weeks, 2 months and 6 months after MRgFUS BBB disruption and AAV delivery (FIG. 5). No evidence of inflammatory response or tissue damage in the sonicated area was detected at any time point in both striatum and cortex (FIGS. 5, 8 and 11-12). Given prior reports of transient inflammation following MRgFUS-mediated BBB disruption alone without delivering any agent to the brain (Jordao et al., 2013; Kovacs et al., 2017), striatal MRgFUS BBB opening alone was performed in additional animals, sacrificed at 3 hours, 24 hours and 48 hours, and confirmed, as expected, an increase in both Iba1 and GFAP staining at these early time points in both striatum and cortex (FIG. 13). Finally, while formal behavioral testing was not conducted, regular gross observation revealed no evidence of abnormal behavior, and periodic monitoring of body weights and food intake showed no evidence of gross metabolic abnormality to suggest poor health.

### Discussion

Minimally invasive or non-invasive therapies are increasingly attractive for diseases traditionally treated with invasive neurosurgical procedures. This is highlighted by the increasing application of radiosurgery for tumors and functional diseases, endovascular therapies for vascular diseases and the recent interest in MRgFUS thalamotomy for essential tremor. Gene therapy in the nervous system remains experimental, but translation of human gene therapy has been led by the neurosurgical community with promising results from human studies. To date, all human CNS gene therapy studies have required direct surgical infusion due to the size of the viral particles and the presence of an intact BBB precluding efficient transfer of viral vectors from the blood to the brain. However, an advantage of direct infusion that would be lost with widespread delivery is the ability to manipulate only a defined population of neurons while avoiding altering off-target cells, which could cause adverse or unintended effects. Previous studies have utilized MRgFUS BBB disruption to deliver viral vectors to the brain, but the efficiency, long-term stability of gene expression and long-term safety of this approach remains unknown. The present results demonstrate that microbubble-facilitated MRgFUS successfully mediated passage of an AAV1/2 viral vectors from the blood stream following IV administration into the targeted striatum without transduction of cells outside the zone of ultrasound delivery. This resulted in efficient and long-term GFP transduction within the targeted striatal neurons for more than one year and caused no obvious toxicity, neuronal loss or long-term inflammatory response as measured by an absence of sustained astrocytic or microglial proliferation at various time points.

MRgFUS follows previous approaches to gene delivery through intravascular administration of viral vectors. The most common has been systemic use of an osmotic agent such as mannitol, which has been shown for many years to transiently open the BBB and has been used successfully in human patients for drug delivery (Neuwelt et al., 1983; Neuwelt et al., 1981; Neuwelt et al., 1991; Neuwelt et al., 1984). Several studies have evaluated the role of mannitol in facilitating global distribution and broad dispersion and AAV-mediated gene expression in the brain (Fu et al., 2007; Fu et al., 2003; Mastakov et al., 2001). In addition, intra-arterial administration of mannitol with an AAV vector allowed limited BBB opening followed by target specific gene expression. Real-time MRI visualization of Gadolinium post BBB disruption was observed immediately upon intra-arterial mannitol administration (Foley et al., 2014). However, the use of mannitol for transient BBB disruption presents two caveats. First, systemic administration of mannitol induces BBB opening throughout the brain. This could be highly desirable for widespread diseases, such as pediatric genetic disorders or infiltrative cancers (Foley et al., 2014; McCarty et al., 2009; Neuwelt && Dahlborg, 1987), but could be problematic when delivering genes that could be therapeutic in one brain region but harmful in other areas, such as the glutamic acid decarboxylase gene for Parkinson's disease (Feigin et al., 2007; Kaplitt et al., 2007; LeWitt et al., 2011). Site-specific delivery of mannitol via an intra-arterial catheter could overcome this problem, but the variability in vascular supply to specific brain regions and the lower limit of the caliber of vessels that can currently be accessed could make reliable transduction of defined targets between individuals with limited off-target transduction more difficult.

One possible concern about MRgFUS BBB disruption is the potential for causing inflammation and tissue damage following brain exposure to the systemic immune system. This could be of particular concern when delivering a potential immunogen such as a viral vector. Earlier studies in rodents have shown evidence of inflammatory reactions following FUS BBB disruption alone, without delivery of any agents. One report, using the same inflammatory markers that we employed here, showed increased microglial activation several hours after BBB disruption, with an astrocytic response observed several days later (Jordao et al., 2013). A recent study has more extensively examined both cellular and humoral mediators of inflammation from 1 to 24 hours following MRgFUS BBB disruption, and reported evidence of a sterile inflammatory response (Kovacs et al., 2017). The goal of the present study was to explore long-term consequences of MRgFUS delivery of gene therapy agents to the brain in order to determine the potential translational therapeutic relevance of this approach, and so inflammatory responses were not examined with such fine detail in the first 24-48 hours following delivery. Nonetheless, there was some evidence of early microgliosis and astrocytosis between 3 hours and 48 hours following MRgFUS BBB disruption alone, consistent with these earlier reports (Kovacs et al., 2017). However, this appeared to resolve over a period of days, and was not evident at 2 week through 6 month time points. No evidence of gross tissue damage or neuronal loss, nor any obvious behavioral abnormalities, to suggest significant toxicity from MRgFUS-mediated delivery of AAV vectors to the striatum under the conditions utilized in the present study, were observed. It should be noted that immune reactions are generally more profound after subsequent exposures, and one potential advantage of gene therapy is the ability to provide long-term benefits from a single treatment. However, should more than one treatment become necessary for particular applications of MRgFUS BBB disruption, regardless of whether this is used for gene transduction or delivery of any other agent, the transient inflammatory reactions observed in both this study and earlier reports could represent a greater concern and should be studied carefully prior to considering clinical applications.

Another issue highlighted by this study is the problem of transduction of systemic organs with intravenous viral vector administration, even if MRgFUS restricts delivery within the brain. This could not only lead to production of potentially bioactive molecules from the gene of interest in undesirable organs, leading to adverse consequences, but could also provoke inflammatory reactions that could result in organ tissue damage. In this study, a dose of 10⁹ vg/g of AAV1/2.GFP administered via a tail vein catheter was sufficient to efficiently transduce the sonicated area in the brain. Under these conditions, GFP expression was not observed in the heart or lungs at any time point. However, the liver revealed GFP expression at 2 weeks, which was lost at later time points. This could represent either a loss of gene expression, loss of cells or both, and extensive analyses of the causes of this observation in the liver are beyond the scope of our current study. However, previous reports have examined the phenomenon of loss of expression in peripheral organs several weeks after AAV transduction. Often this appears to be an immune mediated effect (Bell et al., 2011; Breous et al., 2011; Manno et al., 2006). While GFP is a foreign gene, which could provoke immune responses that might not be observed with delivery of genes encoding native proteins, immune reactions against the AAV vector used to transduce the cells can also cause loss of expression even after transduction has successfully occurred (Bell et al., 2011; Manno et al., 2006). In fact, the loss of GFP expression in the liver several weeks following gene delivery without loss of expression in the brain further supports the likelihood that restoration of brain immune privilege following closure of the BBB was achieved prior to onset of GFP expression. Nonetheless, to both limit off-target effects of gene expression in peripheral organs and to prevent possible immune-mediated tissue injury, development of methods to restrict viral vector transduction of peripheral organs following intravenous delivery may be very important for successful clinical translation of MRgFUS-mediated gene delivery for many CNS disorders.

Furthermore, the rodent device that was employed uses a single transducer element, so the area of focus is rather large compared to what might be achieved with an array of transducers, such as the system currently in use for human lesioning. In one embodiment, direct injection of the gene therapy vectors i employed. In one embodiment, systemic delivery of the gene therapy vector is employed.

The present data supports the use of MRgFUS as a safe and efficient means for non-invasive, stable, focal gene delivery in the mammalian brain. In summary, the study demonstrates that BBB disruption using MRgFUS can be a safe and efficient method for site-specific delivery of viral vectors to the brain, raising the potential for non-invasive human gene therapy. The long-term safety and stability of gene expression reported here supports this approach in the clinic. Since direct infusion of AAV vectors has been safely applied to many human patients for a variety of diseases, and at least one human clinical device was recently FDA approved to perform MRgFUS thalamotomy for essential tremor, all necessary technology is currently available for translation into human studies. The long-term safety and gene expression data supports the continued development of this approach as a potentially viable future option for non-invasive focal CNS gene therapy.

### REFERENCES

Abbott et al., Neurobiol. Dis., 37:13 (2010).
Alonso et al., Mol. Ther. Nucleic Acids, 2:e73 (2013)
Bell et al., Hum. Gene Ther., 22:985 (2011).
Breous et al., Gastroenterology, 141:348-357, 357 e341-343, (2011).
Carty et al., J. Neurosci. Methods. 194:144 (2010).
Elias et al., N. Engl. J. Med., 369:640 (2013)
Elias et al., N. Engl. J. Med., 375:730 (2016).
Feigin et al., Proc. Natl. Acad. Sci. USA, 104:19559 (2007).
Fiandaca et al., Neuroimage, 47:T27 (2009).
Foley et al., J. Control Release, 196:71 (2014).
Fu et al., Gene Ther., 14:1065 (2007).
Fu et al., Mol. Ther., 8:911 (2003).
Hsu et al., PLoS One, 8:e57682 (2013).
Huang et al., Exp. Neurol., 233:350 (2012).
Hynynen et al., Radiology, 220:640 (2001).
Hynynen, Expert Opin. Drug Deliv., 4:27 (2007).
Jeanmonod et al., Neurosurg Focus, 32:E1 (2012).
Jordao et al., Exp. Neurol., 248:16 (2013).
Kaplitt et al., Lancet, 369:2097 (2007).
Kaplitt et al., Nat. Genet., 8:148 (1994).
Kovacs et a., Proc. Natl. Acad. Sci. USA, 114:E75 (2017).
LeWitt et al., Lancet Neurol., 10:309 (2011).
Manno et al., Nat. Med., 12:342 (2006).
Marks et al., Lancet Neurol., 7:400 (2008).
Marks et al., Lancet Neurol., 9:1164 (2010).
Mastakov et al., Mol. Ther., 3:225 (2001).
McCaffrey & Davis, J. Investig. Med., 60:1131 (2012).
McCarty et al., Gene Ther., 16:1340 (2009).
McDannold et al., Ultrasound Med. Biol., 31:1527 (2005).
Mingozzi & High, Curr. Gene Ther., 11:321 (2011).
Morgenstern et al., Methods Mol. Biol., 793:443 (2011).
Nance et al., J. Control Release, 189:123 (2014).
Neuwelt & Dahlborg, J. Neurooncol., 4:195 (1987).
Neuwelt et al., Ann. Intern. Med., 94:449 (1981).
Neuwelt et al., J. Clin. Invest., 64:684 (1979).
Neuwelt et al., J. Clin. Oncol., 9:1580 (1991).
Neuwelt et al., Neurosurgery, 12:662 (1983).
Neuwelt et al., Neurosurgery, 15:362 (1984).
Neuwelt et al., Neurosurgery, 17:419 (1985).
Rafii et al., Alzheimers Dement., 10:571 (2014).
Salegio et al., Adv. Drug Deliv. Rev., 64:598 (2012).
Schuster et al., Front Neuroanat., 8:42 (2014).
Thevenot et al., Hum. Gene Ther., 23:1144 (2012).
Treat et al., Int. J. Cancer, 121:901 (2007).
Treat et al., Ultrasound Med. Biol., 38:1716 (2012).
Tuszynski et al., JAMA Neurol., 72:1139 (2015).
Tuszynski et al., Nat. Med., 11:551 (2005).
Wang et al., Gene Ther., 12:1453 (2005).
Wang et al., Gene Ther., 22:104 (2015).
Wang et al., Sci. Rep., 7:39955 (2017).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A non-invasive method to deliver a prophylactic or therapeutic transgene to one or more regions of a brain or spinal cord of a mammal, comprising: administering to a mammal in need thereof, an amount of a population of microbubbles and an amount of a non-oncolytic recombinant virus or a plasmid comprising the transgene; and applying to one or more regions of the central nervous system of the mammal focused ultrasound in an amount that provides for delivery of the recombinant virus or the plasmid to the one or more regions of the brain or spinal cord.

2. The method of claim 1 wherein the microbubbles comprise a mammalian serum protein.

3. The method of claim 1 wherein the mammal is a human.

4. The method of claim 1 further comprising administering a magnetic resonance imaging (MRI) contrast agent to the mammal.

5. The method of claim 1 wherein the microbubbles and the recombinant virus or the plasmid are concurrently administered.

6. The method of claim 1 wherein a composition comprising the microbubbles and the recombinant virus or the microbubbles and the plasmid is administered.

7. The method of claim 1 wherein the focused ultrasound is applied after the microbubbles and the recombinant virus or the plasmid are administered or wherein the focused ultrasound is applied concurrently with the administration of the microbubbles and the recombinant virus or the plasmid.

8-10. (canceled)

11. The method of claim 1 wherein the transgene encodes a protein, a glycoprotein, a miRNA, or siRNA, wherein the protein is optionally a recombinase.

12. (canceled)

13. The method of claim 1 wherein the transgene is flanked by recombination sites for a recombinase and optionally the mammal is administered the recombinase.

14. (canceled)

15. The method of claim 13 wherein a retrograde virus is employed to deliver a recombinase gene or the transgene is inactivated by subsequent delivery of a recombinase enzyme or a gene encoding a recombinase enzyme which prevents transgene expression.

16-17. (canceled)

18. The method of claim 17 wherein a target sequence for a microRNA is inserted into the transgene and wherein the corresponding microRNA is expressed in an organ or tissue region where transgene expression is undesirable.

19. (canceled)

20. The method of claim 1 wherein the mammal has Parkinson's disease, Alzheimer's disease, depression, dementia, a cardiovascular disease or a cerebrovascular disease.

21. (canceled)

22. The method of claim 1 wherein the virus comprises adeno-associated virus, adenovirus, lentivirus, or herpes simplex virus.

23. The method of claim 1 wherein the virus or the plasmid is encapsulated in or attached to the microbubbles or the plasmid is encapsulated in or attached to a nanoparticle or a liposome.

24. (canceled)

25. The method of claim 1 wherein the focused ultrasound is applied to the striatum, hippocampus, or basal forebrain.

26. A non-invasive method to inhibit, restrict or prevent expression in specific regions in a brain or spinal cord of a mammal, comprising: administering to a mammal in need thereof, an amount of a population of microbubbles and an amount a non-oncolytic recombinant virus or a plasmid comprising a therapeutic or prophylactic transgene and a target sequence for a microRNA inserted into the transgene, wherein the corresponding microRNA is expressed in one or more regions of the brain or spinal cord where transgene expression is undesirable; and applying to the central nervous system of the mammal focused ultrasound in an amount that provides for delivery of the recombinant virus or the plasmid to the central nervous system.

27. The method of claim 26 wherein the virus or the plasmid is in a microparticle or in the microbubbles that is/are disrupted in the ultrasound field or by a specific temperature

28. The method of claim 26 wherein the transgene is flanked by recombination sites for a recombinase and optionally the mammal is administered the recombinase.

29-30. (canceled)

31. A non-invasive method to deliver an anti-cancer agent to a resected portion of a brain or spinal cord of a mammal, comprising: administering to a mammal in need thereof, an amount of an anti-cancer agent that is not an oncolytic virus; and applying to one or more regions at or near the resected portion of the brain or spinal cord of the mammal focused ultrasound in an amount that provides for delivery of the anti-cancer agent.

32. The method of claim 31 wherein the anti-cancer agent comprises an antibody or an antibody fragment that binds an antigen, a chemotherapeutic drug or a virus comprising a therapeutic transgene.

33-34. (canceled)
